(19) United States
(12) Patent Application Publication
(10) Pub. No.: US 2025/0087367 A1
(43) Pub. Date: Mar. 13, 2025

(54) METHOD, APPARATUS, DEVICE AND STORAGE MEDIUM FOR INFORMATION INTERACTION

(71) Applicant: Beijing Zitiao Network Technology Co., Ltd., Beijing (CN)

(72) Inventors: Wei TU, Beijing (CN); Yihang Tang, Beijing (CN)

(21) Appl. No.: 18/882,636

(22) Filed: Sep. 11, 2024

(30) Foreign Application Priority Data
Sep. 11, 2023 (CN) 202311170280.6

(51) Int. Cl. G16H 50/30; G16H 10/60

(52) U.S. Cl. CPC G16H 50/30; G16H 10/60

(57) ABSTRACT

Embodiment of the disclosure provides a method, apparatus, device and storage medium for information interaction. The method includes extracting health information of a first consulting object based at least on interactive information of a user and a digital assistant in at least one health consultation for the first consulting object. The user is associated with the first consulting object. The method also includes updating a health record of the first consulting object with the extracted health information. The method also includes in response to a current health consultation being initiated, generating a reply of the digital assistant to a message entered by the user based on the health record of the first consulting object and the message.

### CROSS-REFERENCE TO RELATED APPLICATION

The present application claims priority to Chinese Patent Application No. 202311170280.6, filed on Sep. 11, 2023 and entitled “Method, Apparatus, Device and Storage Medium for Information Interaction”, the entirety of which is incorporated herein by reference.

### FIELD

Example embodiments of the present disclosure generally relate to the field of computers, and in particular, to a method, apparatus, device, and computer-readable storage medium for information interaction.

### BACKGROUND

With the development of Internet technologies, in terms of health consultation, it is also desirable to enable people to enjoy remote and online services. The online health consultation high-efficiency and low-cost high-quality medical resource supply is improved, the medical institution is helped to expand the radiation range of the medical service, and the consulting object can obtain the medical guidance of the authoritative professional at any time. Currently, some websites, terminal applications, etc., attempt to provide online health consultation services. However, in online health consultation, there is an improvement in interactivity and expertise.

### SUMMARY

In a first aspect of the present disclosure, an information interaction method is provided. The method includes extracting health information of a first consulting object based at least on interactive information of a user and a digital assistant in at least one health consultation for the first consulting object. The user is associated with the first consulting object. The method also includes updating a health record of the first consulting object with the extracted health information. The method also includes in response to a current health consultation being initiated, generating a reply of the digital assistant to a message entered by the user based on the health record of the first consulting object and the message.

In a second aspect of the present disclosure, an apparatus for information interaction is provided. The apparatus includes: a health information extraction module configured to extract health information of a first consulting object based at least on interactive information of a user and a digital assistant in at least one health consultation for the first consulting object; a health record updating module configured to a health record of the first consulting object with the extracted health information, the user being associated with the first consulting object; and a reply generation module configured to in response to a current health consultation being initiated, generate a reply of the digital assistant to a message entered by the user based on the health record of the first consulting object and the message.

In a third aspect of the present disclosure, an electronic device is provided. The device includes at least one processing unit; and at least one memory coupled to the at least one processing unit and storing instructions for execution by the at least one processing unit. The instructions, when executed by the at least one processing unit, cause the device to perform the method of the first aspect.

In a fourth aspect of the present disclosure, a computer-readable storage medium is provided. The medium stores a computer program, and when the computer program is executed by the processor, the method in the first aspect is implemented.

It should be understood that the content described in this section is not intended to limit the key features or important features of the embodiments of the present disclosure, nor is it intended to limit the scope of the present disclosure. Other features of the present disclosure will become readily understood from the following description.

### BRIEF DESCRIPTION OF DRAWINGS

The above and other features, advantages, and aspects of various embodiments of the present disclosure will become more apparent from the following detailed description taken in conjunction with the accompanying drawings. In the drawings, the same or similar reference numbers refer to the same or similar elements, wherein:

FIG. 1 illustrates a schematic diagram of an example environment in which embodiments of the present disclosure can be implemented;

FIG. 2A to FIG. 2G show schematic diagrams of example interfaces for health consultation according to some embodiments of the present disclosure, respectively;

FIG. 3A to FIG. 3C show schematic diagrams of an example interface for generating a health record according to some embodiments of the present disclosure, respectively;

FIG. 3D illustrates a schematic diagram of an example interface presenting a health record according to some embodiments of the present disclosure;

FIG. 4 shows a flowchart of a process for information interaction according to some embodiments of the present disclosure;

FIG. 5 illustrates a block diagram of an apparatus for information interaction according to some embodiments of the present disclosure; and

FIG. 6 illustrates a block diagram of an electronic device in which one or more embodiments of the present disclosure may be implemented.

### DETAILED DESCRIPTION

Embodiments of the present disclosure will be described in more detail below with reference to the accompanying drawings. While certain embodiments of the present disclosure are shown in the accompanying drawings, it should be understood that the present disclosure may be implemented in various forms, and should not be construed as limited to the embodiments set forth herein, but rather, these embodiments are provided for a more thorough and complete understanding of the present disclosure. It should be understood that the drawings and embodiments of the present disclosure are for exemplary purposes only and are not intended to limit the scope of the present disclosure.

In the description of the embodiments of the present disclosure, the terms “including” and the like should be understood to include “including but not limited to”. The term “based on” should be understood as “based at least in part on”. The terms “one embodiment” or “the embodiment” should be understood as “at least one embodiment”. The term “some embodiments” should be understood as “at least some embodiments”. Other explicit and implicit definition may also be included below.

Herein, unless explicitly stated, “responding to A” performs one step and does not imply that this step is performed immediately after “A”, but may include one or more intermediate steps.

It may be understood that the data involved in the technical solution (including but not limited to the data itself, the obtaining, using, storing or deleting of the data) should follow the requirements of the corresponding laws and regulations and related regulations.

It can be understood that before using the technical solutions disclosed in the embodiments of the present disclosure, relevant user should be informed of the types, use ranges, usage scenarios, and the like of the information related to the present disclosure in an appropriate manner according to relevant laws and regulations, and the authorization of the related user may be obtained, wherein the relevant users may include any type of rights body, such as individuals, businesses, and groups.

For example, when an active request of a user is received, prompt information is sent to the related user to explicitly prompt the related user, and the operation requested to be performed will need to obtain and use the information of the related user, so that the related user can autonomously select whether to provide information to software or hardware executing the operation of the technical solution of the present disclosure according to the prompt information.

As an optional but non-limiting implementation, in response to receiving an active request of a related user, a manner of sending prompt information to the related user may be, for example, a pop-up window, and prompt information may be presented in a text manner in the pop-up window. In addition, the pop-up window may further carry a selection control for the user to select “agree” or “not agree” to provide information to the electronic device.

It may be understood that the foregoing notification and obtaining a user authorization process are merely illustrative, and do not constitute a limitation on implementations of the present disclosure, and other manners of meeting related laws and regulations may also be applied to implementations of the present disclosure.

As used herein, the term “model” may learn an association relationship between respective inputs and outputs from training data such that a corresponding output may be generated for a given input after training is complete. The generation of the model may be based on machine learning techniques. Deep learning is a machine learning algorithm that processes inputs and provides corresponding outputs by using a multi-layer processing unit. The neural network model is one example of a deep learning model. As used herein, a “model” may also be referred to as a “machine learning model,” a “learning model,” a “machine learning network,” or a “learning network,” which terms are used interchangeably herein.

In the online health consultation process, the user may input the health information of the consulting object by filling in a form or the like. However, this manner of collecting health information may cause problems such as incomplete information collection, errors in information filling, and large consumed time. Therefore, there is a lack of an efficient solution to efficiently and accurately collect health information and update health records. In addition, health records cover extensive information, various information formats, units and standards may be inconsistent, and therefore data standardization is difficult to achieve. On the other hand, current online health service platforms often only support personal health record management, and lack health record management for a plurality of people or families.

According to an embodiment of the present disclosure, an improved solution for information interaction is provided. In this solution, health information of a first consulting object is extracted based at least on interactive information of a user and a digital assistant in at least one health consultation for the first consulting object. The consulting object is associated with the user. For example, the consulting object may be the user himself or herself, or other consulting object associated with the user. A health record of the first consulting object is updated with the extracted health information. If a current health consultation is initiated, a reply of the digital assistant to a message entered by the user is generated based on the health record of the first consulting object and the message.

In this way, the health record of the consulting object may be updated based on information interaction between the user and the digital assistant, thereby better managing the health information of the consulting object. In addition, since the consulting object may be a certain consulting object associated with the user, the user may manage and update the health record of the plurality of consulting object associated therewith, thereby improving the experience feeling of the health service.

Some example embodiments of the present disclosure will be described in detail below with reference to the accompanying drawings.

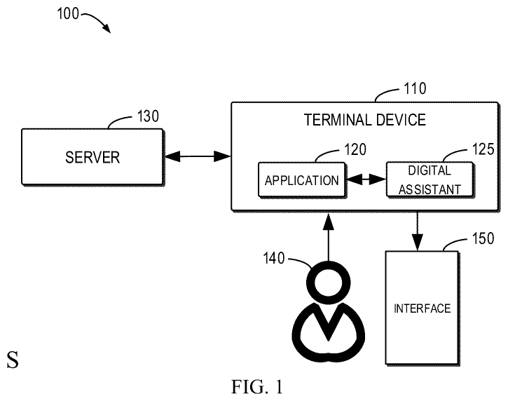

FIG. 1 illustrates a schematic diagram of an example environment 100 in which embodiments of the present disclosure can be implemented. In this example environment 100, an application 120 is installed in the terminal device 110. The user 140 may interaction with the application 120 via the terminal device 110 and/or an attachment device of the terminal device 110.

In some embodiments, the application 120 may be downloaded, installed on the terminal device 110. In some embodiments, the application 120 may also be accessed in other manners, such as web page access. In the environment 100 of FIG. 1, if the application 120 is launched, the terminal device 110 may present the interface 150 of the application 120. The interface 150 may include various user interfaces that may be provided by the application 120. Interface 150 is also sometimes referred to as a client interface.

In embodiments of the present disclosure, the application 120 includes any suitable application capable of providing a health consultation service. In some embodiments, the application 120 may implement a dialog interaction with the user 140 based on the digital assistant 125 to provide a health consultation service to the user 140. In some embodiments, the application 120 may provide other services in addition to the interaction of the digital assistant 125, which is not limited in this respect.

In some embodiments, terminal device 110 may communicate with server 130 to enable provision of services to applications 120 and/or digital assistant 125. The terminal device 110 may be any type of mobile terminal, fixed terminal, or portable terminal, including a mobile phone, a desktop computer, a laptop computer, a notebook computer, a netbook computer, a tablet computer, a media computer, a multimedia tablet, a personal communication system (PCS) device, a personal navigation device, a personal digital assistant (PDA), an audio/video player, a digital camera/camcorder, a television receiver, a radio broadcast receiver, an electronic book device, a gaming device, or any combination of the foregoing, including accessories and peripherals of these devices, or any combination thereof. In some embodiments, the terminal device 110 can also support any type of interface for a user (such as a “wearable” circuit, etc.). Applications 130 may be various types of computing systems/servers capable of providing computing power, including, but not limited to, mainframes, edge computing nodes, computing devices in a cloud environment, and so forth.

It should be understood that the structures and functions of the various elements in the environment 100 are described for exemplary purposes only and do not imply any limitation to the scope of the present disclosure.

In an embodiment of the present disclosure, the digital assistant 125 is configured to interact with a user. An interactive interface, also referred to as an interactive window or a dialog window, of the user and the digital assistant 125 is presented in the client interface. The digital assistant 125, as an intelligent assistant, has an intelligent dialogue and information processing capability. In the interactive interface, the user enters an interactive message, and the digital assistant 125 provides a reply message in response to user input. In application 120, the digital assistant 125 may be launched or awakened in an appropriate manner (e.g., a shortcut, button, or voice) to present an interactive interface with the user. The interaction interface may include an interface element for information interaction, such as an input box, a message list, a message bubble, and the like.

In embodiments of the present disclosure, digital assistant 125 is used to provide services related to health consultation, which may sometimes be referred to as a “digital doctor”. The user may send a health consultation request to the digital assistant 125 by using a simple text or voice input, and may obtain a real-time reply of the digital assistant 125 for medical knowledge science and answer, health consultation, or general health management. In this way, the digital assistant 125 can be used to achieve high efficiency and low cost to improve the supply of high-quality medical resources, help the medical institution to expand the radiation range of the medical service, and enable the user to obtain the guidance of the authoritative professional at any time.

In some embodiments, the digital assistant 125 may determine a reply to the user's question by means of a model. In some embodiments, the model may be trained based on a general corpus and a specialized corpus for the medical domain, enabling actionable, trusted, understandable medical knowledge publicity and health consultation guidance. The dedicated corpus in the medical field may include, for example, national internal and external authoritative clinical guidelines, documents, textbooks, clinical doctor-patient communication question-answer data, and the like. In some embodiments, the training of the medical safety related mechanism can be strengthened during model training, so that the model can have the capability of providing credible health consultation suggestions, and a comprehensive safety test is achieved. In some embodiments, when evaluating the performance of the digital assistant 125, the capability of the digital assistant 125 in terms of communication expression ability and humanistic care and friendliness may also be evaluated, so that the digital assistant 125 can realize the problems of short communication time and insufficient communication between the digital assistant 125 and the user.

The model may be deployed on the terminal device 110 or may be accessed by the terminal device 110. Alternatively, the terminal device 110 may communicate with the model to provide the application management service for the user 140 using the model. For example, the terminal device 110 may send a prompt word input to the model based on the interaction information between the user 140 and the digital assistant 125, and obtain a processing result of the model. Alternatively, or additionally, the digital assistant 125 itself may also be derived based on a model. The digital assistant 125 may process the session message input by the user 140 to obtain a requirement of the user 140, for example, a problem expected to be consulted by the user 140, and the like. The model may be configured locally or independently of terminal device terminal device 110 according to requirements, and the present disclosure is not intended to be limited thereto.

In an embodiment of the present disclosure, in response to the consultation initiating operation, an interactive interface of the user 140 and the digital assistant 125 is provided for the health consultation of the user 140. The consultation initiation operation may be an operation initiated by the user 140 in any suitable manner. The health consultation may be a health consultation with the user 140 as a consulting object, or may be other consulting object specified by the user 140. In the interactive interface between the user 140 and the digital assistant 125, various types of interactive messages may be supported by the user through text or voice input. The voice input may be converted to a text message. The digital assistant 125 determines a corresponding reply based on the text entered by the user.

In some cases, the user 140 may enter a file in a non-text message format for consultation. In an embodiment of the present disclosure, messages supporting a user to enter a multimodal data format are interaction, including a text message format and a non-text message format. The digital assistant 125 can also properly determine the reply when user user 140 enters the file in the non-text message format.

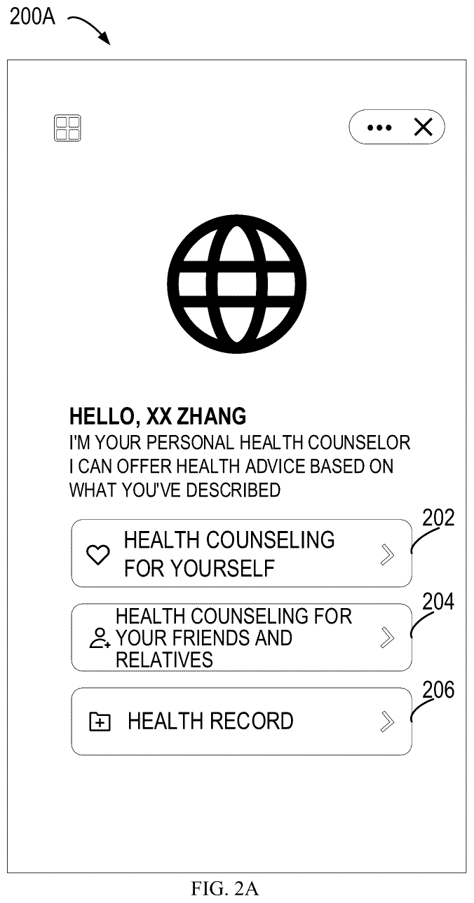

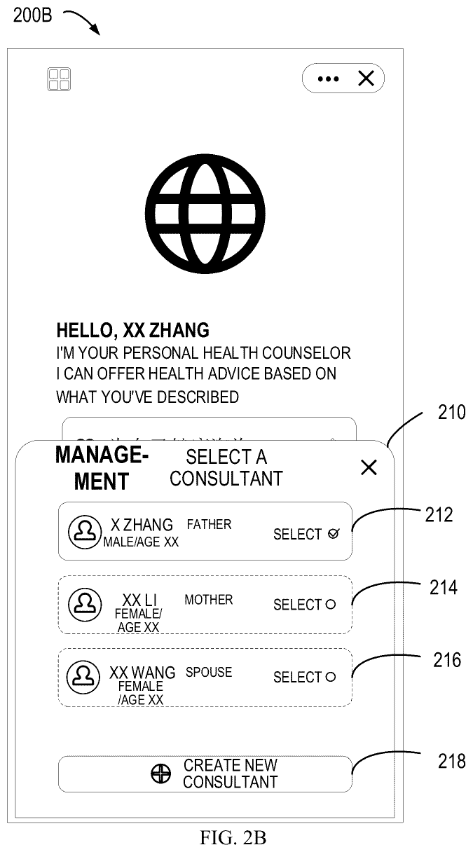

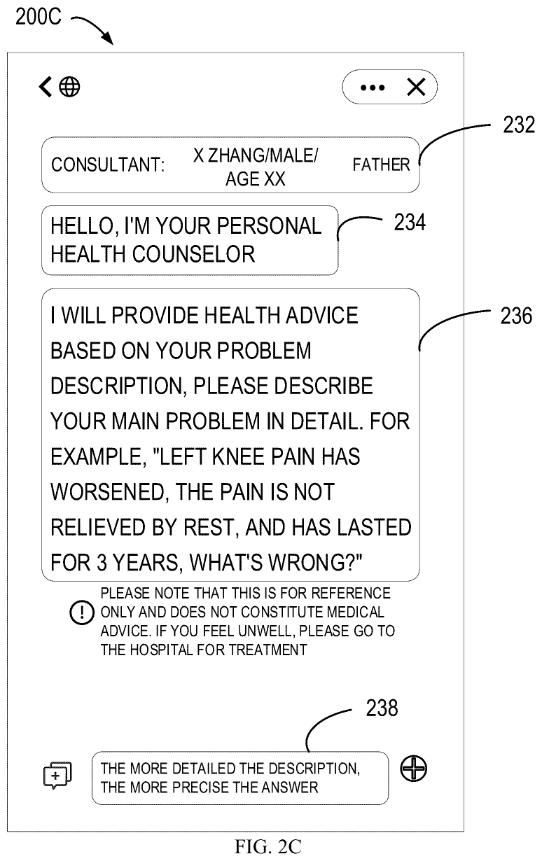

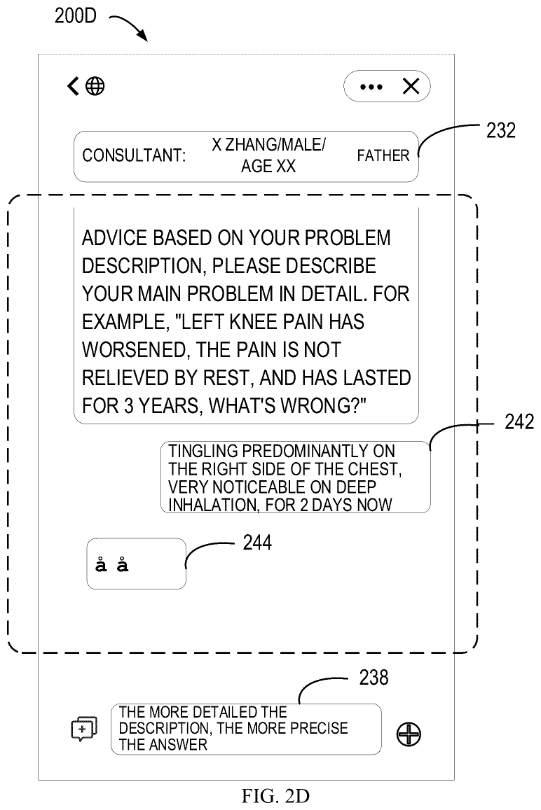

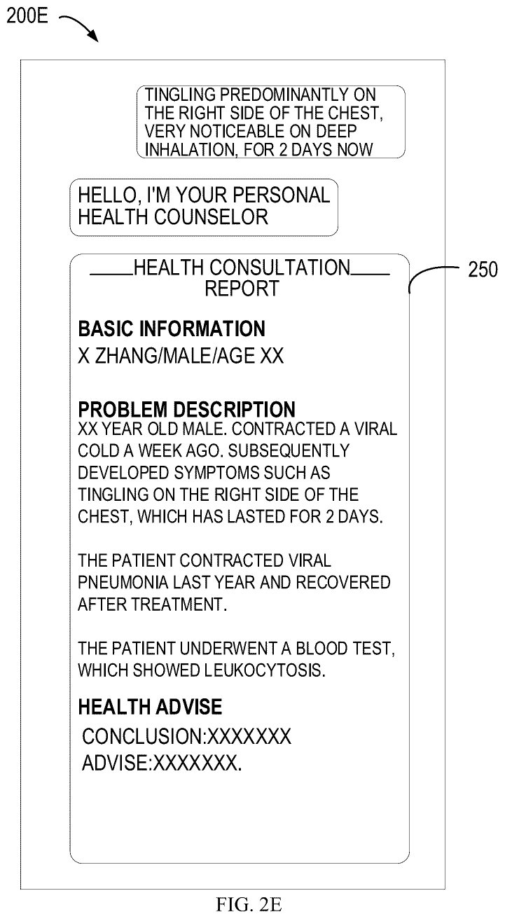

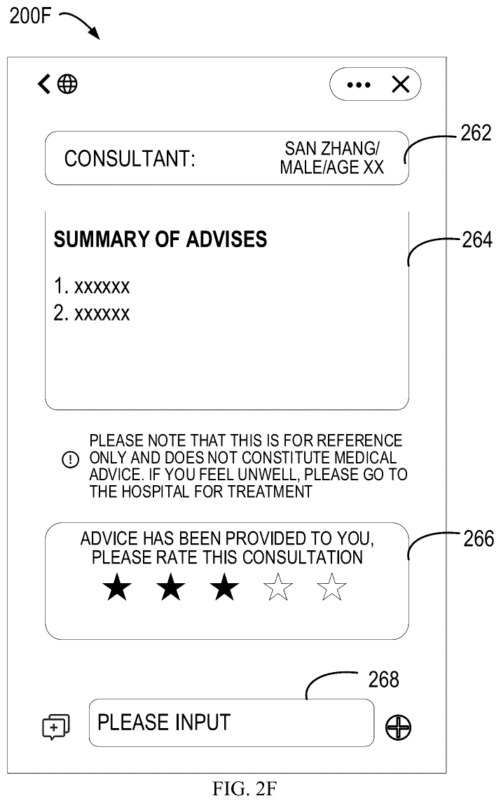

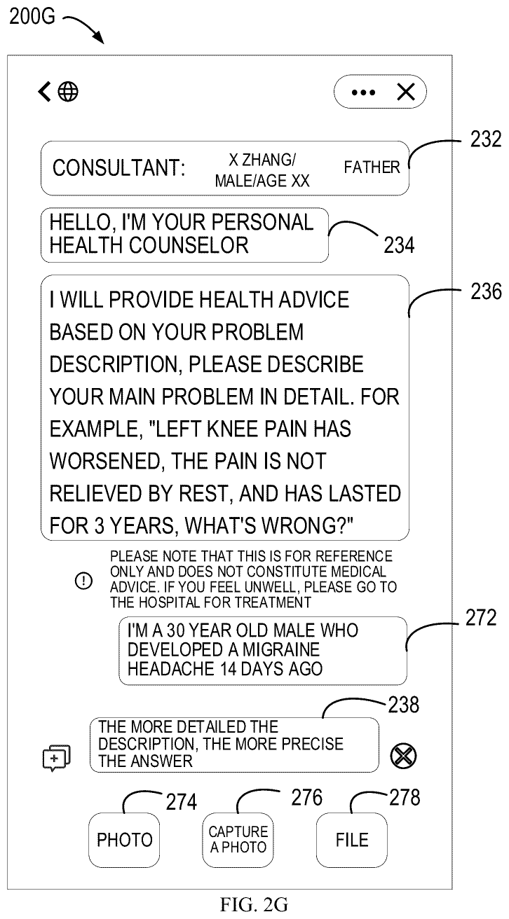

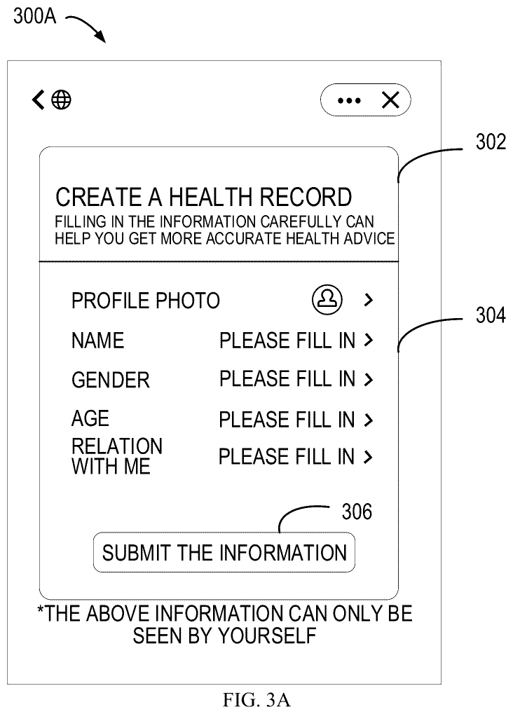

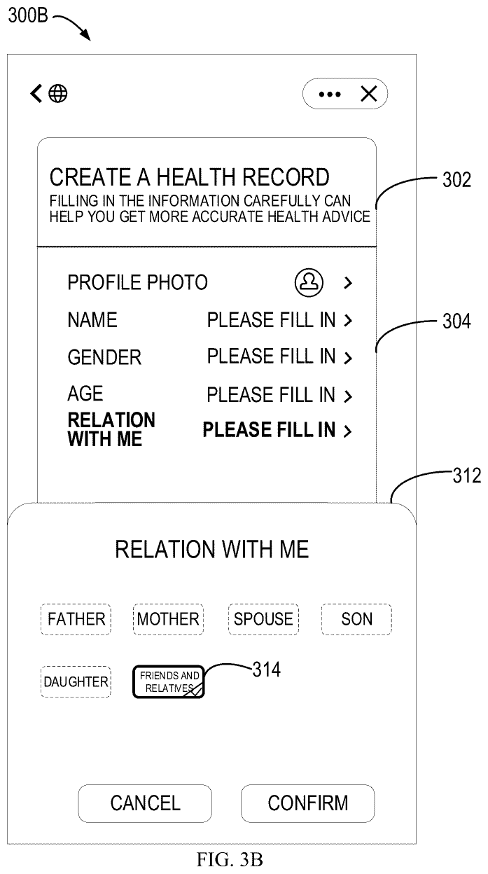

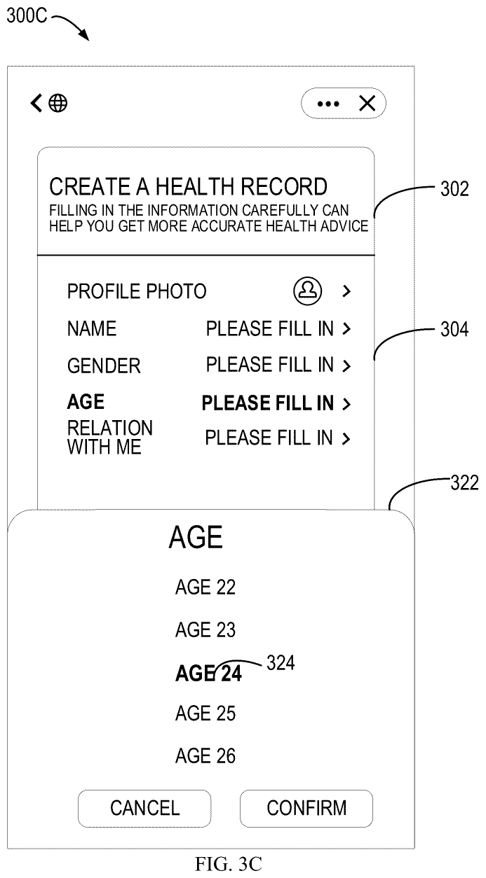

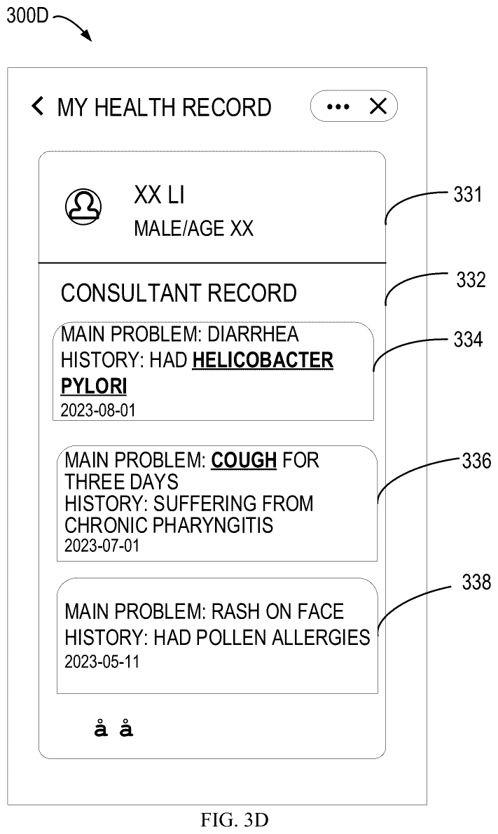

Hereinafter, for a better understanding of the embodiments of the present disclosure, reference will be made to the examples of the information interaction interfaces shown in FIG. 2A to FIG. 2G and FIG. 3A to FIG. 3D. It should be understood that the information interaction interface in these figures is merely an example, and various interface designs may actually exist. Individual graphical elements in the interface may have different arrangements and different visual representations, one or more of which may be omitted or replaced, and one or more other elements may also be present. Embodiments of the present disclosure are not limited in this respect.

As previously mentioned, the digital assistant 125 provides a health consultation service (also referred to as a health consultation service) to the user 140. FIG. 2A illustrates a schematic diagram of an example interface 200A for health consultation in accordance with some embodiments of the present disclosure. The example interface 200A is also referred to herein as an information interaction interface 200A. As shown, the interface 200A is provided with an option 202 for health consulting for the user 140 itself, an option 204 for health consulting for the friends and relatives, and an option 206 for creating a health record.

In some embodiments, if the current health consultation is initiated, a consultation option, such as option 202 and option 204, corresponding to each of the plurality of consulting object will be provided in interface 200A. It should be understood that while in the example of FIG. 2A a single option 204 is presented for a plurality of relatives associated with the user 140, in some embodiments, different options may be presented that correspond to different relatives or consulting object, respectively.

In some embodiments, if the user 140 selects the option 204, the example interface 200B shown in FIG. 2B will be presented. In interface 200B, a consultant management window 210 (also referred to as a consulting object management window) is presented. The user 140 may select a friend or relative who wants to make a consultation in the advisor management window 210. For example, the user 140 may select a corresponding consulting person (i.e., consulting object) from among the options 212, options 214, and options 216 to help. For example, in the illustrated example, the selection 212 is selected, the consulting person to make the consultation is the user's father “X Zhang”. Herein, the advisor presented in the advisor management window 210 is also referred to as a friend advisory of the user 140, or a consulting object or consultant associated with the user 140.

Additionally, or alternatively, in some embodiments, an option 218 may also be presented in the interface 200B for creating a consultant. In this manner, the user 140 may associate a relatives or consulting object associated therewith to an account of the user 140. Therefore, the user 140 may uniformly manage the health consultation or health record of the plurality of friends or consulting object.

It should be understood that while in the example of FIG. 2C, the consultant management window 210 is presented as a pop-up window that at least partially covers the interface 200C, while in some embodiments, the advisor management window 210 may also be presented in other forms. The scope of the present disclosure is not limited in this respect.

In some embodiments, assuming option 218 is selected in the consultant management window 210 of FIG. 2B, the example interface 200C shown in FIG. 2C will be presented. In other words, based on receiving the selection of the consultation option 218 related to the first consulting object (e.g., the consulting object “X Zhang”), the interface 200C is presented. Herein, the selected consulting object is represented by a “first consulting object”. In interface 200C, basic information 232 of the first consulting object is presented. In addition, in the interface 200C, interaction information between the user 140 and the digital assistant 125 is also presented, for example, a dialogue 234, a dialogue 236, and the like. Dialog 234 and dialog 236 may be dialogs sent by digital assistant 125 to user 140 for prompting user 140 for input. Additionally, in the interface 200C, prompt information 238 is also presented to prompt the user 140 to describe in detail the problem that the consultation is desired.

FIG. 2D illustrates an example interface 200D. In interface 200D, user 140 provides an input message 242. The incoming message 242 includes questions that the user 140 wants to consult. In response to the input 242, the digital assistant 125 may generate a reply 244 to the message 242.

In some embodiments, in response to the current health consultation being initiated, a reply to the message 242 by the digital assistant 125 may be generated based on the health record of the first consulting object and the message 242 entered by the user 140. The health record of the first consulting object includes the health information of the first consulting object. The health information of the first consulting object is extracted based at least on the interaction information of the user 140 with the digital assistant 120 in at least one health consultation for the first consulting object. For example, the at least one health consultation for the first consulting object may be all historical health consultation before the current health consultation. As another example, at least one health consultation for the first consulting object may also be a predetermined number of historical health consultation before the current health consultation.

Additionally, or alternatively, in some embodiments, the health information of the first consulting object may be extracted based on at least one of a brief summary of health consultation or an electronic medical record generated in at least one health consultation for the first consulting object, in addition to at least one health consultation for the first consulting object. Herein, the health consultation brief summary refers to summary information of health consultation obtained by summarizing and summarizing the interaction information in the health consultation process. The electronic medical record refers to an electronic record created for the first consulting object that records a disease condition of the consulting object acquired from the health consultation. The health information of the consulting object can be more fully extracted based on the health consultation small knot and the electronic medical record.

Several examples of extracting health information based on at least one health consultation of the first consulting object are described above. In some embodiments, the health record of the first consulting object is updated with the extracted health information. As an example, the health record of the first consulting object may be updated with the occurrence of a health consultation for the first consulting object. In other words, after each health consultation of the first consulting object, the health record of the first consulting object may be updated according to the interaction information of the sub-health consultation.

In some embodiments, the extracted health information is organized in a health record according to a timeline corresponding to at least one health consultation. For example, the health information extracted from each health consultation corresponding to each health consultation may be stored in association with the time of the sub-health consultation. With the continuous health consultation of the user, the corresponding information can be better displayed in the health record, which is convenient for the user to view.

Based on the health record of the first consulting object and the message 242 entered by the user 140, a reply 244 of the digital assistant 125 to the message 242 may be generated. As an example, the reply 244 of the digital assistant 125 may be generated based on the health record of the first consulting object and the message 242 by, for example, a natural language processing technique and a machine learning algorithm. It should be understood that any suitable natural language processing technology or machine learning algorithm may be used to generate the reply of the digital assistant 125, and the embodiments of the present disclosure are not limited in this regard.

The following uses the model to generate the reply 244 as an example for description. As an example, a prompt word input for the model may be generated based on the health record of the first consulting object and the message 242 entered by the user 140. For example, the prompt word input may be a field extracted from the message 242 related to the health of the first consulting object. The generated prompt are input to the model to obtain the output of the model. For example, the output of the model may be a field for health advice or health analysis in connection with the hinting word input. Based on the input of the model, a reply 244 may be generated for the user 140. For example, a statement including the output of the model may be generated as the reply 244 based on the output of the model.

In each dialogue, the health record of the consulting object is used as an input of the model. In this way, the model can provide more accurate, personalized health consultation and health advice according to the specific health condition and demand of the consulting object.

In this way, the digital assistant 125 may continually generate a reply to the message entered by the user 140. Therefore, interaction information, such as multi-round dialogue information, between the user 140 and the digital assistant 125 can be obtained. In some embodiments, based on the interaction information or multi-turn dialog information of the current health consultation, a health consultation report (also referred to herein as a health consultation report) of the current health consultation may be generated, as shown by the health consultation report 250 in the example interface 200E of FIG. 2E. In the health consultation report 250, information such as personal information, question description information, and health recommendations of the first consulting object may be included. In some embodiments, the health consultation report 250 may act as a health consultation small for the current health consultation. The health consultation report 250 may be used to further update the health record of the first consulting object.

In some embodiments, the health record of the consulting object may have a plurality of predetermined fields. Each predetermined field indicates a corresponding information type. The plurality of predetermined fields are also referred to herein as “minimum set fields”. Examples of the plurality of predetermined fields include, but are not limited to: “sternal discomfort”, “sleep disorder”, “polyp”, “inflammation”, “laboratory examination”, etc., and health related terms or terms. The scope of the present disclosure is not limited herein.

In the process of updating the health record of the first consulting object, the extracted health information may be filled into a corresponding field of the plurality of predetermined fields based on the information type indicated by the plurality of predetermined fields. As an example, fields such as “sternum discomfort” may be filled to “sternum discomfort”, and so on.

By setting the plurality of predetermined fields, comprehensive, effective and standard information in the health record can be ensured. By collecting these information, subsequent health analysis and recommendations can be facilitated.

An example process of health consultation is described above in connection with FIGS. 2C-2E. In some embodiments, the user 140 may also evaluate the health consultation. FIG. 2E illustrates an example interface 200E for evaluating health consultation. The interface 200E presents the personal information 262 of the first consulting object, the health information involved in the current health consultation, such as the small knot 264 of the current health consultation, and the evaluation box 266 and the evaluation box 268 for evaluating the current health consultation. The user 140 may score the current health consultation in the evaluation block 266. Further, the user 140 may add a comment to the current health consultation in the evaluation block 268. In this way, the experience feeling of the user can be improved.

As described above, the health information of the first consulting object may be extracted based on the interaction information between the user 140 and the digital assistant 125 in at least one health consultation. In some embodiments, the interaction information between the user 140 and the digital assistant 125 is not limited to dialog information. As an example, the user 140 may also provide other information about the first consulting object to the digital assistant 125.

FIG. 2G illustrates an example interface 200G for providing other information of the first consulting object to the digital assistant 125. In interface 200G, user 140 may not only enter message 272, but user 140 may also utilize option 274, option 276, or option 278 to provide other information of the first consulting object. For example, the user 140 may provide a picture related to the health information of the first consulting object through the option 274, such as various inspection results or inspection reports. For another example, the user 140 may photograph the content related to the health information of the first consulting object through the option 276. For example, the user 140 may take a paper medical record or other examination result or report of the first consulting object through the option 276. As another example, the user 140 may upload a file, such as an electronic medical record, an electronic examination report, etc., regarding the health information of the first consulting object through the option 278.

In some embodiments, the interaction information of the user 140 and the electronic assistant 125 in the current health consultation and the content uploaded by the user 140 such as the picture, the shooting, and the file listed above may be collectively extracted. The health record of the first consulting object may be further updated in the health information extracted in the current health consultation.

Several examples for health consultation and extracting health information based on health consultation and updating a health record are described above in connection with FIGS. 2A-2G. In some embodiments, the user 140 may create a respective health record for a consulting object (e.g., a first consulting object) associated with the user 140. FIG. 3A illustrates a schematic diagram of an example interface 300A for generating a health record, in accordance with some embodiments of the present disclosure. As an example, interface 300A may be presented in response to user 140 selection of option 206 in interface 200A.

As shown, the interface 300A includes prompt information 302 for creating a health record and an information input area 304. In the information input area 304, the user 140 may input information such as a profile phota, name, gender, age, relationship with the user 140, etc., of the current consulting object (e.g., the first consulting object). In addition, an option 306 for submitting information is further provided in the interface 300A for submitting the filled information.

In some embodiments, if the user 140 selects to fill in a relationship with the user 140 (i.e., “Relationship with me”), a separate information window 312 may be presented, as shown by the interface 300B of FIG. 3B. In the information window 312, various predetermined relationship options are presented. For example, in the illustrated example, the user 140 selects the current consulting object from its relationship as a relative relationship 314. It should be understood that the various relationships illustrated in FIG. 3B are merely exemplary and not limiting. In some embodiments, in addition to these common predetermined relationship options, the user 140 may select itself to input other relationship information.

Similarly, in some embodiments, if the user 140 selects to fill in the age of the current consulting object, a separate information window 322 may be presented, as shown by interface 300C of FIG. 3C. In the information window 322, various alternative ages may be presented in a manner such as scrolling presentation. The user 140 may select the age of the current consulting object by, for example, mouse scrolling or the like. As shown, in this example, the age “Age 24” 324 is selected.

It should be understood that while in the examples of FIG. 3B and FIG. 3C, information window 312 and information window 322 are presented as pop-up windows that at least partially cover interfaces 300B and 300C, in some embodiments, information window 312 and information window 322 may also be presented in other forms. The scope of the present disclosure is not limited in this respect.

In a similar manner, the user 140 may input various types of information of the current consulting object, to obtain basic information of the health record of the current consulting object. After creating the health record for the current consulting object (i.e., initialization the health record for the current consulting object), the option for the current consulting object may be presented in a consultant management window 210, such as FIG. 2B. The user 140 may extract the health information of the consulting object by selecting the consulting object and interact with the digital assistant 125 for the consulting object, such as illustrated in FIGS. 2C-2G, and update the health record of the consulting object.

In FIG. 3D, an example interface 300D presenting a health record of a consulting object is shown. The interface 300D includes a region 331 in which basic information or personal information of the consulting object is presented. The interface 300D also includes a region 332 in which the health information of the consulting object is presented. In the illustrated example, extracted health information (e.g., advisory records) for a plurality of health consultation of a consulting object is organized based on a timeline corresponding to the plurality of health consultation. For example, the plurality of consulting records 334, 336, and 338 are presented in the most recent order in the order of the respective health consultation. For another example, in some embodiments, the health record may further present health information or a health change trend (not shown) of the consulting object over a period of time in a form such as a table or a graph. In the embodiments of the present disclosure, the presentation form of the health record is not limited.

In some embodiments, in the health record, at least one entity word may be labeled with a corresponding entry link. As used herein, “entity words” generally refer to terms or words related to medical or health, which typically have a certain medical meaning. As shown in FIG. 3D, an entity word such as “Helicobacter pylori” or “cough” is shown as a bold underline as an example to be labeled with a corresponding entry link. These entity words may be selected or identifier according to any suitable technique or method. Embodiments of the present disclosure are not limited in this regard.

In some embodiments, the entry link may be used to access an entry page corresponding to the entity word. As an example, if the user 140 selects an entity word, the entry page corresponding to the entity word will be presented on the interface 300D in a form such as a pop-up window. Alternatively, the entry page corresponding to the entity word may be presented by jumping to an external page, such as a browser page. On the entry page, a medical meaning or medical interpretation of the entity word may be presented. By presenting the entry link and the entry page of the entity word, the user or the consulting object can quickly and conveniently obtain the medical meaning or interpretation related to the health information, thereby improving the experience feeling of the user.

In some embodiments, an access link (not shown) for the health record may be provided in a page associated with the health record (e.g., interface 300D or other suitable page). As an example, the access link to the health record may be a two-dimensional code or another suitable form of link or hyperlink.

Alternatively, or additionally, access links for interactive information may also be provided in a page (e.g., interface 300D or other suitable page) associated with the health record. As an example, the access link for the interaction information may be a QR code or other suitable form of link or hyperlink for viewing the conversation history of the user 140 with the digital doctor or digital assistant 125.

By providing an access link for a health record or interactive information, the user 140 may view the health record of the consulting object at any time or interactive information such as a historical health consultation dialog. Thus, the user 140 may track the health status of the consulting object.

As mentioned above, the user 140 may be associated with a plurality of consulting object. For example, the user 140 may make a health consultation for different consulting objects associated therewith and generate health records for the respective consulting objects. In some embodiments, the user 140 may manage their respective health records. For example, the health records of the family members may be created and managed separately. The user 140 may switch between health records of different consulting object. The health consultation dialogue or interaction information and the health record of each consulting object will be stored independently. Additionally, if the consulting object currently consulted by the user 140 does not match the currently stored health record, the user 140 may be reminded to switch to the corresponding health record, thereby ensuring the accuracy of the information.

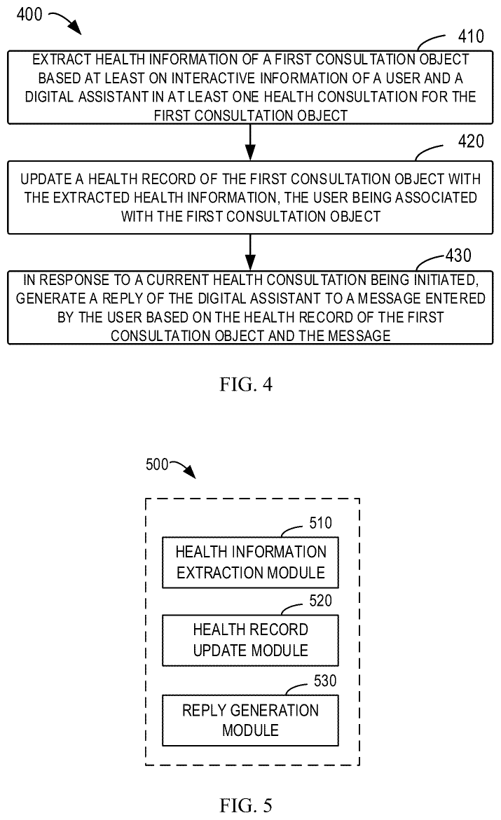

FIG. 4 shows a flowchart of a process 400 for information interaction according to some embodiments of the present disclosure. Process 400 may be implemented at the terminal device 110. The process 400 is described below with reference to FIG. 1.

At block 410, the terminal device 110 extracts health information of a first consulting object based on at least interactive information of the user 140 and the digital assistant 125 in at least one health consultation for the first consulting object. In some embodiments, the terminal device 110 may further extract the health information of the first consulting object based on at least one of a brief summary of health consultation or an electronic medical record generated in at least one health consultation for the first consulting object.

At block 420, the terminal device 110 updates the health record of the first consulting object with the extracted health information. The user is associated with the first consulting object. In some embodiments, the health record has a plurality of predetermined fields. Each predetermined field indicates a corresponding information type. In such embodiment, at block 420, the terminal device 110 may populate the extracted health information into respective fields of the plurality of predetermined fields based on the information type indicated by the plurality of predetermined fields.

At block 430, in response to the current health consultation being initiated, the terminal device 110 generates a reply of the digital assistant 120 to the message 125 based on the health record of the first consulting object and the message entered by the user 140.

In some embodiments, the user 140 is associated with a plurality of consulting object. The plurality of consulting objects includes a first consulting object. Each consulting object has a respective health record. In such an embodiment, the terminal device 110 may provide, in response to the current health consultation being initiated, a consultation option corresponding to each of the plurality of consulting object. At block 430, the terminal device 110 may generate a reply to the message by the digital assistant 125 based on receiving the selection of the consultation option associated with the first consulting object, based on the health record of the first consulting object, and the message entered by the user 140.

In some embodiments, at block 430, the terminal device 110 may generate a prompt word input for a model based on the health record and the message entered by the user 140; provide the generated prompt word input to the model to obtain an output of the model; and generate a reply to the user 140 based on the output of the model.

In some embodiments, the extracted health information is organized in a health record based on a timeline corresponding to at least one health consultation.

In some embodiments, the health record of the first consulting object is updated as the health consultation for the first consulting object occurs.

In some embodiments, in the health record, at least one entity word is labeled with a corresponding word entry link. Each word entry link is used to access a word entry page corresponding to the entity word.

In some embodiments, at least one of: an access link of the health record or an access link of the interaction information is provided in a page associated with the health record.

FIG. 5 is a schematic structural block diagram of an apparatus 500 for information interaction according to some embodiments of the present disclosure. The apparatus 500 may be implemented or included in the terminal device 110. The various modules/components in the apparatus 500 may be implemented by hardware, software, firmware, or any combination thereof.

As shown, the apparatus 500 includes a health information extraction module 510 configured to extract health information of a first consulting object based at least on interactive information of a user and a digital assistant in at least one health consultation for the first consulting object. The apparatus 500 further includes a health record updating module 520 configured to update a health record of the first consulting object with the extracted health information. The user is associated with the first consulting object. The apparatus 500 further includes a reply generation module 530 configured to in response to a current health consultation being initiated, generate a reply of the digital assistant to a message entered by the user based on the health record of the first consulting object and the message.

In some embodiments, the health information extraction module 510 is configured to extract the health information of the first consulting object further based on at least one of: a brief summary of health consultation or an electronic medical record generated in the at least one health consultation for the first consulting object.

In some embodiments, the health record has a plurality of predetermined fields. Each predetermined field indicates a corresponding information type. In such embodiments, the health record updating module 520 may be configured to fill the extracted health information into a corresponding field of the plurality of predetermined fields based on information types indicated by the plurality of predetermined fields.

In some embodiments, in the health record, the extracted health information is organized based on a timeline corresponding to the at least one health consultation.

In some embodiments, a user is associated with a plurality of consulting objects. The plurality of consulting objects includes the first consulting object and each consulting object has a respective health record. In such embodiment, the reply generation module 530 includes: a consultation option providing module configured to in response to the current health consultation being initiated, provide respective consultation options corresponding to the plurality of consulting objects; and a first reply generation module configured to: based on receiving a selection of a consultation option associated with the first consulting object, generate the reply of the digital assistant to the message based on the health record of the first consulting object and the message entered by the user.

In some embodiments, the reply generation module 530 includes a prompt word input generation module configured to generate a prompt word input for a model based on the health record and the message entered by the user; a model output obtaining module configured to provide the generated prompt word input to the model to obtain an output of the model; and a second reply generation module configured to generate the reply to the user based on the output of the model.

In some embodiments, the health record of the first consulting object is updated with an occurrence of a health consultation for the first consulting object.

In some embodiments, in the health record, at least one entity word is labeled with a corresponding word entry link. Each word entry link is used for accessing a word entry page corresponding to the entity word.

In some embodiments, at least one of the following is provided in a page associated with the health record: an access link of the health record, or an access link of the interactive information.

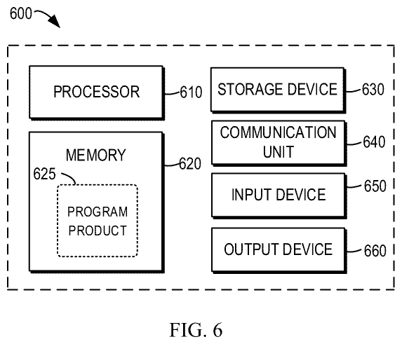

FIG. 6 illustrates a block diagram of an electronic device 600 in which one or more embodiments of the present disclosure may be implemented. It should be understood that the electronic device 600 illustrated in FIG. 6 is merely exemplary and should not constitute any limitation on the functionality and scope of the embodiments described herein. The electronic device 600 shown in FIG. 6 may be configured to implement the terminal device 110 in FIG. 1 or the apparatus 500 in FIG. 5.

As shown in FIG. 6, the electronic device 600 is in the form of a general-purpose computing device. Components of the electronic device 600 may include, but are not limited to, one or more processors or processing units 610, a memory 620, a storage device 630, one or more communication units 640, one or more input devices 650, and one or more output devices 660. The processing unit 610 may be an actual or virtual processor and capable of performing various processes according to programs stored in the memory 620. In multiprocessor system, a plurality of processing units execute computer-executable instructions in parallel to improve parallel processing capabilities of electronic device 600.

Electronic device 600 typically includes a plurality of computer storage media. Such media may be any available media accessible to the electronic device 600, including, but not limited to, volatile and non-volatile media, removable and non-removable media. The memory 620 may be volatile memory (e.g., registers, caches, random access memory (RAM)), non-volatile memory (e.g., read-only memory (ROM), electrically erasable programmable read-only memory (EEPROM), flash memory), or some combination thereof. Storage device 630 may be a removable or non-removable medium and may include a machine-readable medium, such as a flash drive, magnetic disk, or any other medium, which may be capable of storing information and/or data and may be accessed within electronic device 600.

The electronic device 600 may further include additional removable/non-removable, volatile/non-volatile storage media. Although not shown in FIG. 6, a disk drive for reading or writing from a removable, nonvolatile magnetic disk (e.g., a “floppy disk”) and an optical disk drive for reading or writing from a removable, nonvolatile optical disk may be provided. In these cases, each drive may be connected to a bus (not shown) by one or more data media interface. The memory 620 may include a computer program product 625 having one or more program modules configured to perform various methods or actions of various embodiments of the present disclosure.

The communication unit 640 is configured to communicate with another electronic device through a communication medium. Additionally, the functionality of components of the electronic device 600 may be implemented in a single computing cluster or a plurality of computing machines capable of communicating over a communication connection. Thus, the electronic device 600 may operate in a networked environment using logical connections with one or more other servers, network personal computers (PCs), or another network node.

The input device 650 may be one or more input devices such as a mouse, a keyboard, a trackball, or the like. The output device 660 may be one or more output devices, such as a display, a speaker, a printer, or the like. The electronic device 600 may also communicate with one or more external devices (not shown) through the communication unit 640 as needed, external devices such as storage devices, display devices, etc., communicate with one or more devices that enable a user to interact with the electronic device 600, or communicate with any device (e.g., a network card, a modem, etc.) that enables the electronic device 600 to communicate with one or more other electronic devices. Such communication may be performed via an input/output (I/O) interface (not shown).

According to example embodiments of the present disclosure, there is provided a computer-readable storage medium having computer-executable instructions stored thereon, wherein the computer-executable instructions are executed by a processor to implement the method described above. According to an exemplary embodiment of the present disclosure, a computer program product is further provided, the computer program product being tangibly stored on a non-transient computer readable medium and comprising computer executable instructions, the computer executable instructions being executed by a processor to implement the method described above.

Aspects of the present disclosure are described herein with reference to flowcharts and/or block diagrams of methods, apparatuses, devices, and computer program products implemented in accordance with the present disclosure. It should be understood that each block of the flowchart and/or block diagram, and combinations of blocks in the flowcharts and/or block diagrams, may be implemented by computer readable program instructions.

These computer-readable program instructions may be provided to a processing unit of a general purpose computer, special purpose computer, or other programmable data processing apparatus to produce a machine, such that the instructions, when executed by a processing unit of a computer or other programmable data processing apparatus, produce means to implement the functions/acts specified in the flowchart and/or block diagram. These computer-readable program instructions may also be stored in a computer-readable storage medium that cause the computer, programmable data processing apparatus, and/or other devices to function in a particular manner, such that the computer-readable medium storing instructions includes an article of manufacture including instructions to implement aspects of the functions/acts specified in the flowchart and/or block diagram(s).

The computer-readable program instructions may be loaded onto a computer, other programmable data processing apparatus, or other apparatus, such that a series of operational steps are performed on a computer, other programmable data processing apparatus, or other apparatus to produce a computer-implemented process such that the instructions executed on a computer, other programmable data processing apparatus, or other apparatus implement the functions/acts specified in the flowchart and/or block diagram block or blocks.

The flowchart and block diagrams in the figures show architecture, functionality, and operation of possible implementations of systems, methods, and computer program products according to various implementations of the present disclosure. In this regard, each block in the flowchart or block diagram may represent a module, program segment, or portion of an instruction that includes one or more executable instructions for implementing the specified logical function. In some alternative implementations, the functions noted in the blocks may also occur in a different order than noted in the figures. For example, two consecutive blocks may actually be performed substantially in parallel, which may sometimes be performed in the reverse order, depending on the functionality involved. It is also noted that each block in the block diagrams and/or flowchart, as well as combinations of blocks in the block diagrams and/or flowchart, may be implemented with a dedicated hardware-based system that performs the specified functions or actions, or may be implemented in a combination of dedicated hardware and computer instructions.

Various implementations of the present disclosure have been described above, which are exemplary, not exhaustive, and are not limited to the implementations disclosed. Many modifications and variations will be apparent to those of ordinary skill in the art without departing from the scope and spirit of the various implementations illustrated. The selection of the terms used herein is intended to best explain the principles of the implementations, the practical application, or improvements to the technology in the marketplace, or to enable others of ordinary skill in the art to understand the various embodiments disclosed herein.

What is claimed is:

1. A method for information interaction, comprising:
extracting health information of a first consulting object based at least on interactive information of a user and a digital assistant in at least one health consultation for the first consulting object;
updating a health record of the first consulting object with the extracted health information, the user being associated with the first consulting object; and
in response to a current health consultation being initiated, generating a reply of the digital assistant to a message entered by the user based on the health record of the first consulting object and the message.

2. The method of claim 1, wherein extracting the health information of the first consulting object comprises:
extracting the health information of the first consulting object further based on at least one of: a brief summary of health consultation or an electronic medical record generated in the at least one health consultation for the first consulting object.

3. The method of claim 1, wherein the health record has a plurality of predetermined fields, each predetermined field indicating a corresponding information type, and wherein updating the health record of the first consulting object comprises:
filling the extracted health information into a corresponding field of the plurality of predetermined fields based on information types indicated by the plurality of predetermined fields.

4. The method of claim 1, wherein in the health record, the extracted health information is organized based on a timeline corresponding to the at least one health consultation.

5. The method of claim 1, wherein the user is associated with a plurality of consulting objects including the first consulting object and each consulting object having a respective health record, wherein generating a reply of the digital assistant to the message comprises:
in response to the current health consultation being initiated, providing respective consultation options corresponding to the plurality of consulting objects; and
based on receiving a selection of a consultation option associated with the first consulting object, generating the reply of the digital assistant to the message based on the health record of the first consulting object and the message entered by the user.

6. The method of claim 1, wherein generating a reply of the digital assistance to the message comprises:
generating a prompt word input for a model based on the health record and the message entered by the user;
providing the generated prompt word input to the model to obtain an output of the model; and
generating the reply to the user based on the output of the model.

7. The method of claim 1, wherein a health record of the first consulting object is updated with an occurrence of a health consultation for the first consulting object.

8. The method of claim 1, wherein in the health record, at least one entity word is labeled with a corresponding word entry link, and each word entry link is used for accessing a word entry page corresponding to the entity word.

9. The method of claim 1, wherein at least one of the following is provided in a page associated with the health record: an access link of the health record, or an access link of the interactive information.

10. An electronic device comprising:
at least one processing unit; and
at least one memory coupled to the at least one processing unit and storing instructions for execution by the at least one processing unit, the instructions, when executed by the at least one processing unit, causing the device to perform a method comprising:
extracting health information of a first consulting object based at least on interactive information of a user and a digital assistant in at least one health consultation for the first consulting object;
updating a health record of the first consulting object with the extracted health information, the user being associated with the first consulting object; and
in response to a current health consultation being initiated, generating a reply of the digital assistant to a message entered by the user based on the health record of the first consulting object and the message.

11. The electronic device of claim 10, wherein extracting the health information of the first consulting object comprises:
extracting the health information of the first consulting object further based on at least one of: a brief summary of health consultation or an electronic medical record generated in the at least one health consultation for the first consulting object.

12. The electronic device of claim 10, wherein the health record has a plurality of predetermined fields, each predetermined field indicating a corresponding information type, and wherein updating the health record of the first consulting object comprises:
filling the extracted health information into a corresponding field of the plurality of predetermined fields based on information types indicated by the plurality of predetermined fields.

13. The electronic device of claim 10, wherein in the health record, the extracted health information is organized based on a timeline corresponding to the at least one health consultation.

14. The electronic device of claim 10, wherein the user is associated with a plurality of consulting objects including the first consulting object and each consulting object having a respective health record, wherein generating a reply of the digital assistant to the message comprises:
in response to the current health consultation being initiated, providing respective consultation options corresponding to the plurality of consulting objects; and
based on receiving a selection of a consultation option associated with the first consulting object, generating the reply of the digital assistant to the message based on the health record of the first consulting object and the message entered by the user.

15. The electronic device of claim 10, wherein generating a reply of the digital assistance to the message comprises:
generating a prompt word input for a model based on the health record and the message entered by the user;
providing the generated prompt word input to the model to obtain an output of the model; and
generating the reply to the user based on the output of the model.

16. The electronic device of claim 10, wherein a health record of the first consulting object is updated with an occurrence of a health consultation for the first consulting object.

17. The electronic device of claim 10, wherein in the health record, at least one entity word is labeled with a corresponding word entry link, and each word entry link is used for accessing a word entry page corresponding to the entity word.

18. The electronic device of claim 10, wherein at least one of the following is provided in a page associated with the health record: an access link of the health record, or an access link of the interactive information.

19. A non-transitory computer readable storage medium having a computer program stored thereon, the computer program, when executed by a processor, implements a method comprising:
extracting health information of a first consulting object based at least on interactive information of a user and a digital assistant in at least one health consultation for the first consulting object;
updating a health record of the first consulting object with the extracted health information, the user being associated with the first consulting object; and
in response to a current health consultation being initiated, generating a reply of the digital assistant to a message entered by the user based on the health record of the first consulting object and the message.

20. The computer readable storage medium of claim 19, wherein extracting the health information of the first consulting object comprises:
extracting the health information of the first consulting object further based on at least one of: a brief summary of health consultation or an electronic medical record generated in the at least one health consultation for the first consulting object.
